(19) United States
(12) Patent Application Publication
(10) Pub. No.: US 2008/0255452 A1
(43) Pub. Date: Oct. 16, 2008

(54) Methods and Apparatus For Performing Enhanced Ultrasound Diagnostic Breast Imaging

(75) Inventor: Robert Randall Entrekin, Kirkland, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/576,214

(22) PCT Filed: Sep. 22, 2005
(86) PCT No.: PCT/IB05/53130

§ 371 Date: Mar. 28, 2007

Related U.S. Application Data
(60) Provisional application No. 60/614,383, filed on Sep. 29, 2004.

(51) Int. Cl. A61B 8/14

(52) U.S. Cl. 600/444

(57) ABSTRACT

A method for performing enhanced ultrasound diagnostic breast imaging includes using first and second compression plates (62,64) configured for receiving and compressing a breast between the same. The breast extends from a chest wall (118) of a patient at a proximate end to a nipple at a distal end. Portions of the breast proximate the nipple and proximate lateral edges of the breast are in non-contact with the second compression plate during breast compression. An ultrasound transducer array (68) moves along a path to scan the breast, the ultrasound transducer array being disposed adjacent a side of the second plate (64) opposite the breast. Image data representative of the breast is acquired as the ultrasound transducer array (68) traverses the path. Acquiring image data includes using electronic beam steering with the ultrasound transducer array to acquire image data in either or both (i) a portion (116) of the breast proximate the chest wall and (ii) a portion of the breast in non-contact with the second plate (122).

### CROSS REFERENCE TO RELATED CASES

This application is related to U.S. Pat. No. 6,682,484, entitled “Compression Plate For Diagnostic Breast Imaging” and U.S. Pat. No. 6,530,885, entitled “Spatially Compounded Three Dimensional Ultrasonic Images”, assigned to the assignee of the present disclosure and incorporated herein by reference in their entirety.

### BACKGROUND OF THE INVENTION

The present disclosure generally relates to medical diagnostic imaging systems, and more particularly, to methods and apparatus for performing enhanced ultrasound diagnostic breast imaging.

Automated three-dimensional (3D) breast ultrasound scanning is similar to mammography, where a breast is compressed between two planar, semi-rigid surfaces or plates. Since the compression plates are flat, a linear array transducer is used so that the transducer face intimately contacts a surface of one of the compression plates. Intimate contact facilitates acoustic coupling of the transducer and the compression plate. In addition, the compression plates are substantially parallel, as in X-ray mammography. Furthermore, the known methods of automated 3D breast ultrasound scanning use linear array transducers to acquire rectangular images perpendicular to the compression plate. The rectangular images are then used to reconstruct a rectangular 3D volume.

However, the automated 3D breast ultrasound scanning methods discussed above fail to image an entire volume of the breast since a compressed breast is not rectangular in shape. The outer edges of the breast, including the nipple, are curved. Accordingly, the curved outer edges of the breast cannot be imaged because they are not in acoustic contact with the compression plate. In one known method, a conformal gel pad or a water bag provides coupling to these curved areas, however the use of the conformal gel pad or water bag is cumbersome. In another method, a moveable “nipple support platform” is used to improve an acoustic contact with the nipple, however, such a support platform is awkward and time consuming to use in practice.

Another problem caused by rectilinear volume scanning is that some breast tissue immediately adjacent to the chest wall of a patient is not visualized, since it cannot be pulled into the space between the compression plates. In another known method, a linear array transducer is mechanically tilted at an angle of approximately fifteen degrees (˜15 degrees) to improve a visualization of the chest wall. However, such a method fails to solve the acoustic contact problem at the curved edges of the breast. It is also unsuitable if the breast needs to have uniform compression thickness, as in the case of combined X-ray and ultrasound imaging.

### SUMMARY OF THE INVENTION

Accordingly, an improved method and apparatus for performing enhanced ultrasound diagnostic breast imaging for overcoming the problems in the art is desired.

According to one embodiment of the present disclosure, a method for performing enhanced ultrasound diagnostic breast imaging includes using first and second compression plates configured for receiving and compressing a breast between the same. The breast extends from a chest wall of a patient at a proximate end to a nipple at a distal end. A portion of the breast proximate the nipple is in non-contact with the second compression plate during breast compression. An ultrasound transducer array moves along a path to scan the breast, the ultrasound transducer array being disposed adjacent a side of the second plate opposite the breast. Image data representative of the breast is acquired as the ultrasound transducer array traverses the path. Acquiring image data includes using electronic beam steering with the ultrasound transducer array to acquire image data in either or both (i) a portion of the breast proximate the chest wall and (ii) a portion of the breast immediately behind the nipple that corresponds to the portion of the breast in non-contact with the second plate.

### BRIEF DESCRIPTION OF THE DRAWINGS

FIG. 1 is a cross-sectional view of a two pass linear scan for obtaining a conventional, non-steered rectangular image;

FIG. 2 is a perspective view of an exposure area of the non-steered linear scan in FIG. 1;

FIG. 3 is a cross-sectional view of a two pass steered linear scan for obtaining a steered linear parallelogram shaped image;

FIG. 4 is a partial block diagram view of an ultrasound diagnostic imaging system for enhanced ultrasound diagnostic breast imaging with 3D spatially steered electronic beam scanning according to an embodiment of the present disclosure;

FIG. 5 is a cross-sectional view of a two pass scan for obtaining a 3D spatially steered trapezoidal image of a breast according to one embodiment of the present disclosure;

FIG. 6 is perspective view of an exposure area of the 3D spatially steered trapezoidal scan in FIG. 5 according to one embodiment of the present disclosure; and

FIG. 7 is a cross-sectional view of a two pass scan for obtaining a 3D spatially steered and compounded trapezoidal image of a breast according to another embodiment of the present disclosure.

### DESCRIPTION OF THE PREFERRED EMBODIMENTS

In the figures, like reference numerals refer to like elements. In addition, it is to be noted that the figures are not drawn to scale.

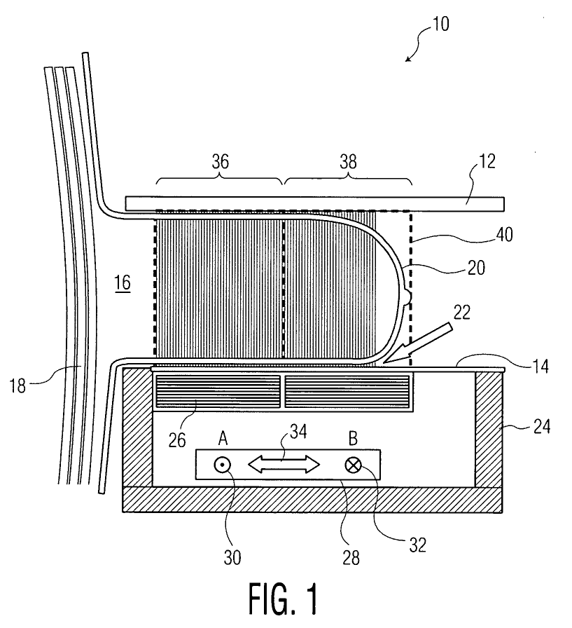

FIG. 1 is a cross-sectional view of a portion of an ultrasound diagnostic breast imaging system that uses a two pass linear scan for obtaining a conventional, non-steered rectangular volumetric image. That is, the embodiment of FIG. 1 uses non-steered rectangular images to acquire 3D volumes. As a result, the two-pass (A and B) 3D scan with conventional, non-steered rectangular image frames fails to image the curved area near the nipple and the tissue adjacent to the chest wall, as discussed further below.

As shown in FIG. 1, the ultrasound diagnostic breast imaging system 10 includes a first compression plate 12 and a second compression plate 14. The first and second plates are configured for receiving a breast 16 and further adapted for compressing the breast between the first and second plates. The breast 16 extends from a chest wall 18 of a patient at a proximate end to a nipple 20 at a distal end. During breast compression, a portion of the breast proximate the nipple is in non-contact with the second compression plate 14, wherein the region of loss of contact is indicated by reference numeral 22.

The second compression plate 14 is mounted on a top surface of a housing 24. An ultrasound transducer array 26 is disposed within the housing 24 adjacent a side of the second plate 14 opposite the breast 16. The transducer array 26 is acoustically coupled to the second plate, for example, using a suitable acoustic coupling liquid. In addition, the ultrasound transducer array is coupled to a mechanical motion system 28. The mechanical motion system 28 can include any suitable scanning mechanism and jig, as are known in the art, and is configured for moving the transducer array 26 along a path to scan the breast.

Further with respect to FIG. 1, a two pass linear scan is used for obtaining a conventional, non-steered rectangular volumetric image. That is, the mechanical motion system 28 operates to move the transducer array along a path that includes first and second passes, as indicated by the circle and dot 30 and the circle and “x” 32. The motion system 28 traverses between the first and second passes as indicated by the arrow 34. As shown, the first pass includes a portion of the path that extends in a vertical direction perpendicular to and out of a plane of the figure. Similarly, the second pass includes a portion of the path that extends in a vertical direction perpendicular to and into the plane of the figure. Image data representative of the breast is then acquired as the ultrasound transducer array traverses the path. Acquiring image data for the two-pass 3D scan of FIG. 1 uses conventional, non-steered rectangular image frames to image the breast, but fails to image the curved area 20 near the nipple and the tissue adjacent to the chest wall 18. A first portion of ultrasound imaging along the first pass is indicated by reference numeral 36. A second portion of ultrasound imaging along the second pass is indicated by reference numeral 38. However, note that a portion of breast 16 is not subject to the ultrasound imaging as a result of the loss of contact in region 22. The potential total area of ultrasound imaging is generally indicated by reference numeral 40.

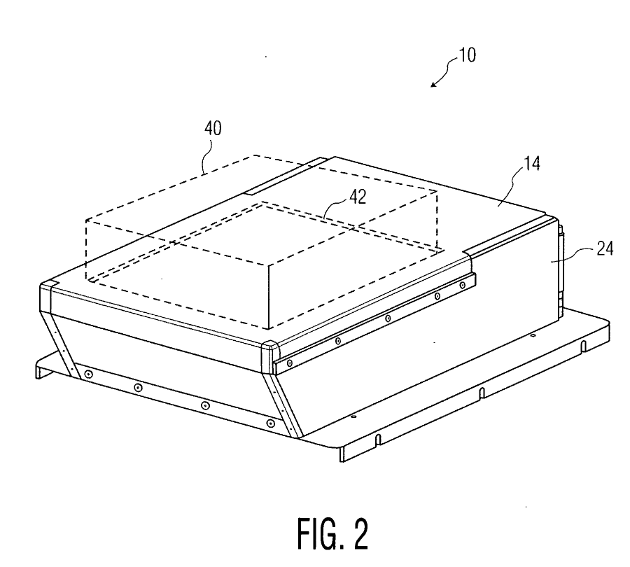

FIG. 2 is a perspective view of a volumetric exposure area 40 of the two-pass 3D non-steered linear scan of FIG. 1. As illustrated, the exposure area 40 comprises a rectangular volume, extending through a peripheral shape 42 within the second plate 14.

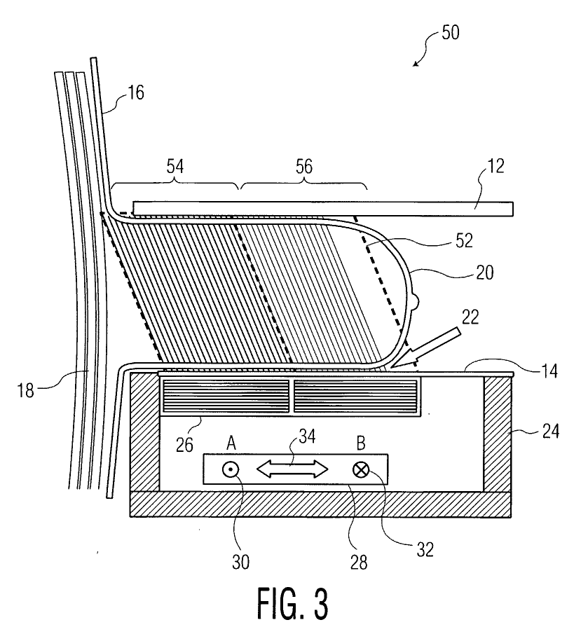

FIG. 3 is a cross-sectional view of a portion of an ultrasound breast imaging system that uses a two pass steered linear scan for obtaining a steered linear image corresponding to a parallelogram volume shaped image. The portion of the ultrasound breast imaging system 50 of FIG. 3 is similar to that of FIG. 1, with the following differences. Image data representative of the breast is acquired as the ultrasound transducer array traverses the path. Acquiring image data for the two-pass 3D scan of FIG. 3 uses a steered parallelogram image frames to image the breast, but fails to image the curved area 20 near the nipple. In other words, the two-pass (A and B) 3D scan with steered parallelogram image frames images a portion of the breast tissue adjacent to the chest wall 18 but fails to image the curved area 20 of the breast near the nipple. Moreover, the steered linear parallelogram image acquires more tissue adjacent to the chest wall, but at the expense of less tissue visualized near the nipple. Conversely, the ultrasound beams could be steered the other direction to see more tissue near the nipple, but at the expense of seeing less tissue near the chest. As illustrated, the exposure area 52 comprises a parallelogram shaped volume that includes a first pass 54 and a second pass 56.

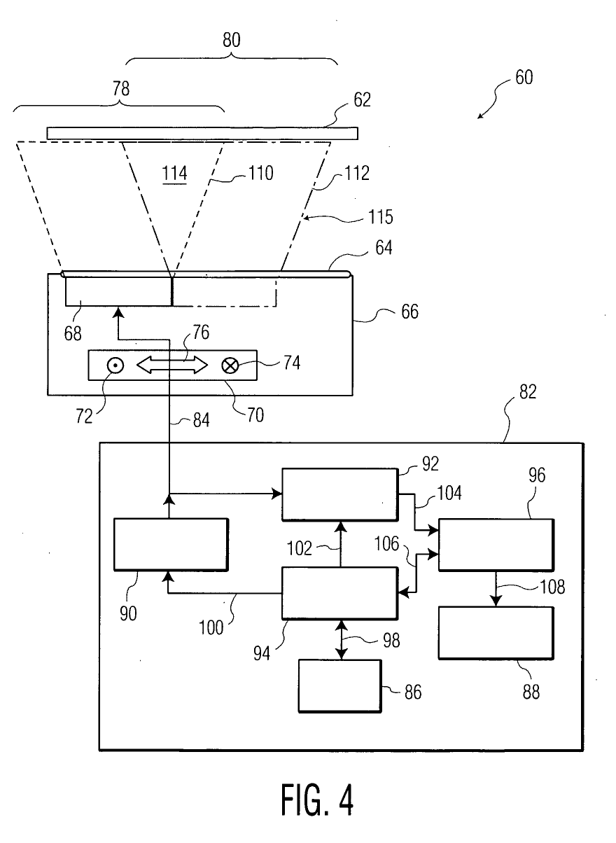

FIG. 4 is a partial block diagram view of an ultrasound diagnostic imaging system 60 for enhanced ultrasound diagnostic breast imaging with 3D spatially steered electronic beam scanning according to an embodiment of the present disclosure. The ultrasound diagnostic breast imaging system 60 includes a first compression plate 62 and a second compression plate 64.

The second compression plate 64 is mounted on a top surface of a housing 66. An ultrasound transducer array 68 is disposed within the housing 66 adjacent a side of the second plate 64 opposite a breast to be imaged. The transducer array 68 is acoustically coupled to the second plate, for example, using a suitable acoustic coupling liquid. In addition, the ultrasound transducer array is coupled to a mechanical motion system 70. The mechanical motion system 70 can include any suitable scanning mechanism and jig, as are known in the art, and is configured for moving the transducer array 68 along a path to scan the breast to be imaged.

Ultrasound diagnostic breast imaging system 60 includes a control electronics unit 82. Ultrasound transducer array 68 couples to the control electronics unit 82 via a signal line 84. The control electronics unit 82 includes and/or interfaces with an input/output device 86 (such as a keyboard, mouse, or the like) and a display device 88, the control electronics unit providing imaging data signals to the video display for visual display. The control electronics unit 82 may further provide ultrasound image data to other devices (not shown), such as a printer, a mass storage device, computer network (i.e., for remote data storage, analysis, and/or display), etc., via data signal transmissions suitable for use by the destination device. In one embodiment, the control electronics unit 82 further includes a transmitter 90 (e.g. a transmit beamformer), digital beamformer 92 (e.g., a receive beamformer), a system controller 94, and an image processor 96.

The system controller couples to the I/O device 86 via signal line 98. The system controller 94 also provides appropriate transmit beamformer control signals to transmitter 90 via signal line 100. The transmit beamformer control signals are configured for providing the desired beam steering by the ultrasound transducer array as discussed further herein. Responsive to the transmit beamformer control signals, transmitter 90 provides corresponding ultrasound transducer control signals to ultrasound transducer array 68 via signal line 84.

In addition, the system controller 94 also provides appropriate receive beamformer control signals to digital beamformer 92 via signal line 102. The receive beamformer control signals are configured for providing a desired beamforming according to the embodiments of the present disclosure, as discussed further herein. Digital beamformer 92 provides ultrasound image data to image processor 96 via signal line 104. Furthermore, system controller 94 couples to image processor 96 via signal line 106. Responsive to control signals from system controller 94 and responsive to ultrasound image data from digital beamformer 92, image processor 96 provides image data to display device 88 via signal line 108, the image data being suitable for use by display device 88. The components of electronic unit 82 can include any suitable components known in the art for carrying out various functions as discussed herein.

Ultrasonic diagnostic breast imaging system 60 performs ultrasonic spatial compounding of volumetric image information in accordance with the embodiments of the present disclosure. Array transducer 68 transmits beams at different angles over an image field as denoted by the dashed trapezoids 110 and 112. Each trapezoid can include, for example, two or three groups of scanlines with the scanlines of each group being steered at a different angle relative to the array transducer. By appropriately steering the groups of scanlines, compounding of component image frames can be used to make up a trapezoidal image frame.

The transmission of ultrasound beams is controlled by transmitter 90. Transmitter 90 controls the phasing and time of actuation of each of the elements of the array transducer 68 so as to transmit each beam from a predetermined origin along the array and at a predetermined angle or steering direction, and focus. The echoes returned from along each scanline are received by the elements of the array, digitized as by analog to digital conversion (not shown), and coupled to a digital beamformer 92. The digital beamformer 92 delays and sums the echoes from the array elements to form a sequence of focused, coherent digital echo samples along each scanline. The transmitter 90 and beamformer 92 are operated under control of system controller 94, which in turn is responsive to the settings of controls of a user interface 86 operated by the user of the ultrasound system. The system controller 94 controls the transmitter 90 to transmit the desired number of scanline groups at the desired angles, focuses, transmit energies and frequencies. The system controller 94 also controls the digital beamformer 92 to properly delay and combine the received echo signals for the apertures and image depths used.

In accordance with the embodiments of the present disclosure, the spatially compounded image data is presented in a three dimensional display format by image processor 96, wherein the image processor includes a volume image rendering processor. Image data from a volumetric region which has undergone spatial compounding, either B mode or Doppler data, is processed by volume image rendering into a 3D display presentation. The rendering is controlled by rendering control signals selected by the user interface 86 and applied to the processor 96 by the system controller 94. The rendering control signals can precondition the processor 96 to render Doppler or tissue signal information, for instance, and/or to render the image data with opacity weighting which will enable flow to be viewed through a volume of tissue, for example. A Cineloop memory (not shown) can also be used in support of the volume rendering processor.

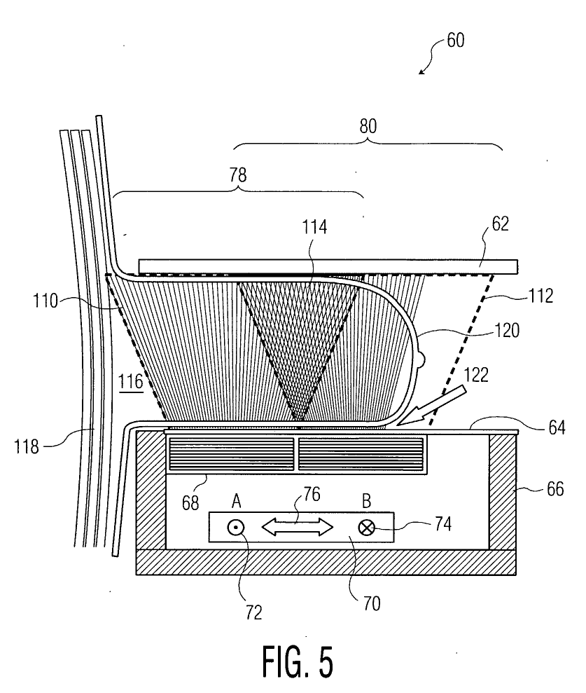

Referring now to FIG. 5, a breast scanning portion of ultrasound diagnostic breast imaging system 60 is schematically illustrated. The breast 114 to be scanned is first retained between two compression plates 62 and 64. In one embodiment the lower compression plate 64 is fixed in location and the upper compression plate 62 is movable to apply a downward compression force which retains the breast. The compressed breast is scanned by ultrasound transducer 68 located below the lower compression plate 64. The transducer 68 scans the breast by articulation of the transducer in two dimensions by a mechanical motion system 70. It will be appreciated that the breast scanning portion of FIG. 5 could also be constructed in an inverted configuration. That is, the ultrasound transducer could scan the breast from above an upper compression plate and either of the compression plates could move to apply the compressive force.

In one embodiment of the present disclosure, the lower compression plate 64 is formed by a thin polymeric sheet which is held under tension in at least one dimension. By using tension, the lower compression plate can be made of a very thin polymeric sheet which is highly transmissive to ultrasound. The tension applied to the sheet provides significant rigidity to the compression plate, a rigidity which, for a thin sheet, can be virtually entirely determined by the amount of tension applied to the sheet. The tension applied can be of any force up to the tensile strength of the polymeric sheet.

As discussed, FIG. 5 is a cross-sectional view of a two pass scan for obtaining a 3D spatially steered trapezoidal image of a breast according to one embodiment of the present disclosure. The two passes include an overlapping center portion of the breast as indicated by reference numeral 114. As shown in FIG. 5, the first and second plates are configured for receiving a breast 116 and further adapted for compressing the breast between the first and second plates. The breast 116 extends from a chest wall 118 of a patient at a proximate end to a nipple 120 at a distal end. During breast compression, a portion of the breast proximate the nipple is in non-contact with the second compression plate 64, wherein the region of loss of contact is indicated by reference numeral 122.

Further with respect to FIGS. 4 and 5, a two pass linear scan is used for obtaining a steered trapezoidal volumetric image. That is, the mechanical motion system 70 operates to move the transducer array along a path that includes first and second passes, as indicated by the circle and dot 72 and the circle and “x” 74. The motion system 70 traverses between the first and second passes as indicated by the arrow 76. As shown, the first pass includes a portion of the path that extends in a vertical direction perpendicular to and out of a plane of the figure. Similarly, the second pass includes a portion of the path that extends in a vertical direction perpendicular to and into the plane of the figure. Image data representative of the breast is then acquired as the ultrasound transducer array traverses the path. Acquiring image data for the two-pass 3D scan of FIG. 5 uses steered trapezoidal image frames to image the breast. Accordingly, the two-pass 3D scan images the curved area 120 near the nipple and the tissue adjacent to the chest wall 118.

In other words, the two-pass (A and B) 3D scan with a trapezoidal linear image of FIG. 5 can acquire more tissue adjacent to the chest wall and near the nipple simultaneously, even with some loss of contact due to curvature at the edge of the breast. The embodiment of FIG. 5 provides for obtaining a maximum field of view at a fastest acquisition speed. A first portion of the ultrasound imaging along the first pass is indicated by reference numeral 78. A second portion of the ultrasound imaging along the second pass is indicated by reference numeral 80.

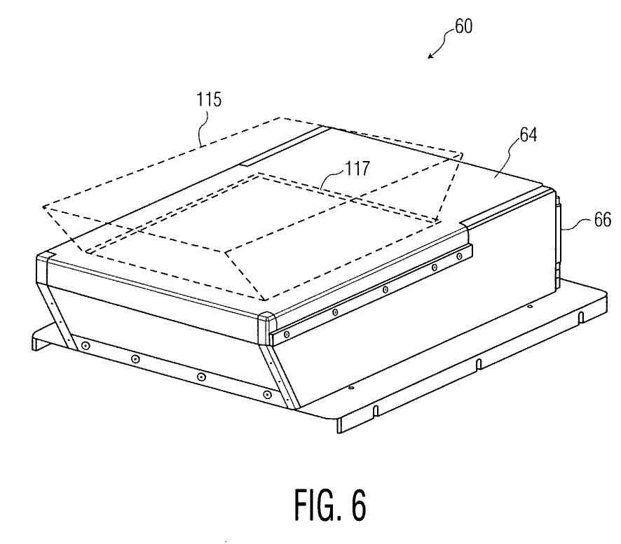

FIG. 6 is a perspective view of a volumetric exposure area 115 of the two-pass 3D spatially steered trapezoidal linear scan of FIG. 5 according to one embodiment of the present disclosure. As illustrated, the exposure area 115 comprises a trapezoidal volume, extending through a peripheral shape 117 within the second plate 64.

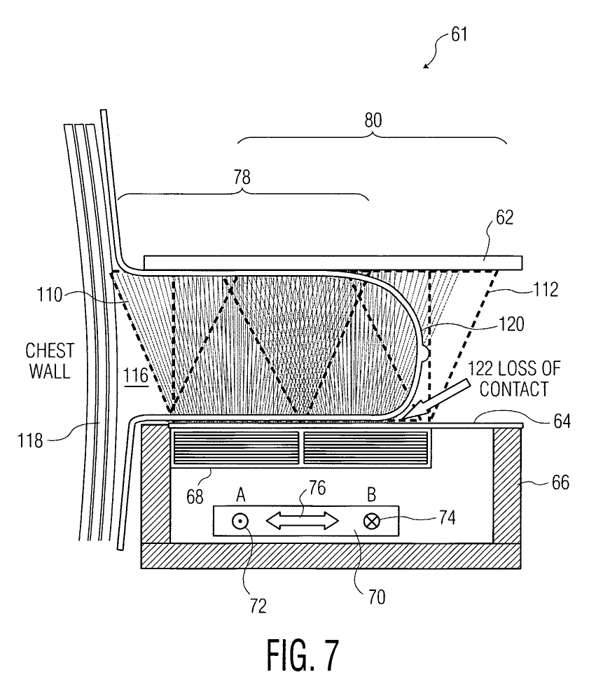

FIG. 7 is a cross-sectional view of a two pass scan for obtaining a 3D spatially steered and compounded trapezoidal image of a breast according to another embodiment of the present disclosure. The embodiment of FIG. 7 is similar to that of FIG. 5 with the following differences. Acquiring image data for the two-pass 3D scan of FIG. 7 uses steered and compounded trapezoidal image frames to image the breast. Accordingly, the two-pass 3D scan images the curved area 120 near the nipple and the tissue adjacent to the chest wall 118.

Co-pending U.S. patent application Ser. No. 09/335,058 and 09/435,118 describe apparatus and methods for performing real time spatial compounding of ultrasonic diagnostic images. Spatial compounding is an imaging technique in which ultrasound image data of a given target that has been obtained from multiple vantage points or look directions are combined into a single compounded image by combining the data for example by linearly or nonlinearly averaging or filtering. The compounded image typically shows lower speckle and better specular reflector delineation than conventional ultrasound images produced from a single look direction. With respect to FIG. 7, the array transducer 68 transmits beams at different angles over an image field as denoted by the dashed trapezoids 110 and 112.

In other words, the two-pass (A and B) 3D scan with a spatially steered and compounded linear image of FIG. 7 can acquire the maximum tissue adjacent to the chest wall and near the nipple simultaneously, even with some loss of contact due to curvature at the edge of the breast. The embodiment of FIG. 7 provides for obtaining a maximum field of view with a highest image quality.

As discussed herein, the embodiments of the present disclosure utilize electronic beam steering to acquire image data of curved areas of breast tissue not in acoustic contact with the compression plate, and much more of the tissue near the chest wall than achieved with prior known methods. The embodiments of the present disclosure include methods for using electronic beam steering to acquire a larger, more complete view of a compressed breast during automated 3D breast ultrasound scans. The embodiments of the present disclosure provide an improvement over the current art, which misses tissue in the areas near the chest wall and behind the nipple.

The embodiments of the present disclosure provide ways to utilize electronic beam steering to acquire a more complete 3D volume of the whole breast. Features of the embodiments include the providing of a linear array transducer in contact with a substantially planar compression plate. In addition, a mechanical scanning system is provided to automatically translate the linear array parallel to the compression plate in a direction perpendicular to the image plane. Furthermore, the embodiments use electronic beam steering in the image plane to acquire trapezoidal and/or spatially compounded images to increase the volume of breast tissue visualized, as compared with the use of a linear array with a non-steered rectangular image or a simple steered linear image.

According to one embodiment, a method for performing enhanced ultrasound diagnostic breast imaging includes providing a first compression plate and a second compression plate. The first and second plates are configured for receiving a breast and further adapted for compressing the breast between the first and second plates. The breast extends from a chest wall of a patient at a proximate end to a nipple at a distal end. In addition, during breast compression, a portion of the breast proximate the nipple is in non-contact with the second compression plate. In one embodiment, the first compression plate and the second compression plates are substantially parallel during compressing of the breast.

The method further includes moving an ultrasound transducer array along a path to scan the breast. The ultrasound transducer array is disposed adjacent a side of the second plate opposite the breast, and further being acoustically coupled to the second plate. Image data representative of the breast is then acquired as the ultrasound transducer array traverses the path. Acquiring image data includes using electronic beam steering with the ultrasound transducer array to acquire image data in either or both (i) a portion of the breast proximate the chest wall and (ii) a portion of the breast immediately behind the nipple that corresponds to the portion of the breast in non-contact with the second plate.

In one embodiment, the ultrasound transducer array comprises a two-dimensional matrix transducer array. In another embodiment, the ultrasound transducer array comprises a linear transducer array. In addition, moving the ultrasound transducer array along the path can include, for example, moving the array by articulation of the transducer in two dimensions. In one embodiment, the path includes one or more passes for scanning a breast. For example, in practice, scanning could vary from one pass to four passes, and can include scanning overlapping portions of the breast, depending upon the size of the compressed breast under examination. In addition, scanning could include selecting different scan areas, which could also include the same number or a different number of passes, based upon the size of the compressed breast. In another embodiment, the path includes two passes along an overlapping center portion of the breast. Furthermore, in another embodiment, moving the ultrasound transducer array can further comprise automatically translating the ultrasound transducer array parallel to the second plate in a direction perpendicular to an image plane of the acquired image data. In yet another embodiment, the diagnostic ultrasound breast imaging system includes a single large transducer array for implementing a one pass scan. Still further, another embodiment uses a small transducer with a one-pass scan for only a small region of interest, instead of the whole breast.

In another embodiment, acquiring image data further includes electronic beam steering configured for a one or more pass, three dimensional scan that acquires spatially steered trapezoidal linear image data. In other words, the electronic beam steering comprises using the electronic beam steering to acquire image data of trapezoidal volume images of the breast. In another embodiment, acquiring image data further includes electronic beam steering configured for a one or more pass, three dimensional scan that acquires spatially steered and compounded trapezoidal linear image data. That is, the electronic beam steering comprises using the electronic beam steering to acquire image data of spatially compounded trapezoidal volume images of the breast.

The method further includes processing the image data, via a suitable processor, to form a three dimensional representation of the breast. In yet another embodiment, the method includes transmitting the image data to a location remote from the acquisition location and processing the transmitted image data to form a three dimensional representation of the breast.

In another embodiment, a diagnostic ultrasound imaging system for enhanced diagnostic breast imaging, comprises first and second compression plates. The first and second plates are configured for receiving a breast and adapted for compressing the breast between the first and second plates. The system further includes an ultrasound transducer array disposed adjacent a side of the second plate opposite the breast. A suitable means is provided for moving the ultrasound transducer array along a path to scan the breast.

The diagnostic ultrasound imaging system further includes a system controller for acquiring image data representative of the breast as the ultrasound transducer array traverses the path. The system controller uses electronic beam steering with the ultrasound transducer array to acquire image data in either or both (i) a portion of the breast proximate the chest wall and (ii) a portion of the breast immediately behind the nipple that corresponds to the portion of the breast in non-contact with the second plate.

In one embodiment, the system controller uses electronic beam steering configured for a one or more pass, three dimensional scan that acquires spatially steered trapezoidal linear image data. Additionally, the use of electronic beam steering further comprises acquiring image data of trapezoidal volume images of the breast. In another embodiment, the system controller uses electronic beam steering configured for a one or more pass, three dimensional scan that acquires spatially steered and compounded trapezoidal linear image data. Furthermore, the use of electronic beam steering further comprises acquiring image data of spatially compounded trapezoidal volume images of the breast.

A processor processes the image data to form a three dimensional representation of the breast. In one embodiment, a means for transmitting the image data to a location remote from the acquisition location, and a processor at the remote location is used for processing of the transmitted image data to form a three dimensional representation of the breast. In addition, the moving means moves the ultrasound transducer array by automatically translating the ultrasound transducer array parallel to the second plate in a direction perpendicular to an image plane of the acquired image data.

In another embodiment, a diagnostic ultrasound imaging system for performing enhanced diagnostic breast imaging includes a first compression plate and a second compression plate. The first and second plates are configured for receiving a breast and adapted for compressing the breast between the first and second plates. An ultrasound transducer array is disposed adjacent to a side of the second plate opposite the breast and is acoustically coupled to the second plate. A translation stage moves the ultrasound transducer array along a path to scan the breast. In addition, a controller acquires image data representative of the breast as the ultrasound transducer array traverses the path.

The controller uses electronic beam steering with the ultrasound transducer array to acquire image data in either or both (i) a portion of the breast proximate the chest wall and (ii) a portion of the breast immediately behind the nipple that corresponds to a portion of the breast in non-contact with the second plate. Additionally, acquiring image data includes electronic beam steering configured for a two pass, three dimensional scan that acquires one of (a) spatially steered trapezoidal linear image data or (b) spatially steered and compounded trapezoidal linear image data.

Although only a few exemplary embodiments have been described in detail above, those skilled in the art will readily appreciate that many modifications are possible in the exemplary embodiments without materially departing from the novel teachings and advantages of the embodiments of the present disclosure. For example, the embodiments of the present disclosure enhance applications of whole breast ultrasound in the field of screening and/or diagnosis of breast cancer. In addition, the embodiments have been discussed herein with respect to a portion of the breast immediately behind the nipple as corresponding to a portion of the breast in non-contact with the second plate. Although the area of non-contact proximate the nipple is clinically very significant, it has been used herein as an example. The same benefits of the present embodiments could be obtained for any other aspect of the breast where there is incomplete contact. For example, the lateral edges of the breast, when compressed, bulge outward. Accordingly, with the lateral edges there is some tissue that, due to the rounded shape of the edge, is not in contact with the bottom breast support (i.e., the second plate). If the scanning transducer is rotated 90 degrees and moved front-to-back instead of right-to-left, the same improvement in visualizing the lateral margins of the breast (as previously discussed with respect to visualizing the nipple and chest wall) would occur.

Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A method for performing enhanced ultrasound diagnostic breast imaging, comprising:
providing a first compression plate;
providing a second compression plate, the first and second plates being configured for receiving a breast and adapted for compressing the breast between the first and second plates, the breast extending from a chest wall of a patient at a proximate end to a nipple at a distal end, further wherein portions of the breast proximate the nipple and proximate lateral edges of the breast are in non-contact with the second compression plate during breast compression;
moving an ultrasound transducer array along a path to scan the breast, the ultrasound transducer array being disposed adjacent a side of the second plate opposite the breast; and
acquiring image data representative of the breast as the ultrasound transducer array traverses the path, wherein acquiring image data includes using electronic beam steering with the ultrasound transducer array to acquire image data in either or both (i) a portion of the breast proximate the chest wall and (ii) a portion of the breast in non-contact with the second plate.

2. The method of claim 1, wherein acquiring image data further includes electronic beam steering configured for a one or more pass, three dimensional scan that acquires spatially steered trapezoidal linear image data.

3. The method of claim 1, wherein acquiring image data further includes electronic beam steering configured for a one or more pass, three dimensional scan that acquires spatially steered and compounded trapezoidal linear image data.

4. The method of claim 1, wherein the electronic beam steering comprises using the electronic beam steering to acquire image data of trapezoidal volume images of the breast.

5. The method of claim 1, wherein the electronic beam steering comprises using the electronic beam steering to acquire image data of spatially compounded trapezoidal volume images of the breast.

6. The method of claim 1, wherein the ultrasound transducer array is acoustically coupled to the second plate.

7. The method of claim 1, wherein moving the ultrasound transducer array along the path includes moving by articulation of the transducer in two dimensions.

8. The method of claim 1, wherein the path includes two passes along an overlapping center portion of the breast.

9. The method of claim 1, wherein the first compression plate and the second compression plates are substantially parallel during compressing of the breast.

10. The method of claim 1, wherein the ultrasound transducer array comprises a two-dimensional matrix transducer array.

11. The method of claim 1, wherein the ultrasound transducer array comprises a linear transducer array.

12. The method of claim 1, further comprising:
processing the image data to form a three dimensional representation of the breast.

13. The method of claim 1, further comprising:
transmitting the image data to a location remote from the acquisition location, and processing of the transmitted image data to form a three dimensional representation of the breast.

14. The method of claim 1, wherein moving the ultrasound transducer array further comprises automatically translating the ultrasound transducer array parallel to the second plate in a direction perpendicular to an image plane of the acquired image data.

15. A method for performing enhanced ultrasound diagnostic breast imaging, comprising:
providing a first compression plate;
providing a second compression plate, the first and second plates being configured for receiving a breast and adapted for compressing the breast between the first and second plates, the breast extending from a chest wall of a patient at a proximate end to a nipple at a distal end, further wherein portions of the breast proximate the nipple and proximate lateral edges of the breast are in non-contact with the second compression plate during breast compression;
providing an ultrasound transducer array disposed adjacent a side of the second plate opposite the breast, the ultrasound transducer array further being acoustically coupled to the second plate;
moving the ultrasound transducer array along a path to scan the breast; and
acquiring image data representative of the breast as the ultrasound transducer array traverses the path, wherein acquiring image data includes using electronic beam steering with the ultrasound transducer array to acquire image data in either or both (i) a portion of the breast proximate the chest wall and (ii) a portion of the breast in non-contact with the second plate, and wherein acquiring image data further includes electronic beam steering configured for a one or more pass, three dimensional scan that acquires at least one selected from the group consisting of (a) spatially steered trapezoidal linear image data and (b) spatially steered and compounded trapezoidal linear image data.

16. A diagnostic ultrasound imaging system for enhanced diagnostic breast imaging, comprising:
a first compression plate;
a second compression plate, wherein the first and second plates are configured for receiving a breast and adapted for compressing the breast between the first and second plates, the breast extending from a chest wall of a patient at a proximate end to a nipple at a distal end, further wherein portions of the breast proximate the nipple and proximate lateral edges of the breast are in non-contact with the second compression plate during breast compression;
an ultrasound transducer array disposed adjacent a side of the second plate opposite the breast;
means for moving the ultrasound transducer array along a path to scan the breast; and
means for acquiring image data representative of the breast as the ultrasound transducer array traverses the path, wherein the means for acquiring image data uses electronic beam steering with the ultrasound transducer array to acquire image data in either or both (i) a portion of the breast proximate the chest wall and (ii) a portion of the breast in non-contact with the second plate.

17. The system of claim 16, wherein the means for acquiring image data further uses electronic beam steering configured for a one or more pass, three dimensional scan that acquires spatially steered trapezoidal linear image data.

18. The system of claim 16, wherein the means for acquiring image data further uses electronic beam steering configured for a one or more pass, three dimensional scan that acquires spatially steered and compounded trapezoidal linear image data.

19. The system of claim 16, wherein using electronic beam steering further comprises acquiring image data of trapezoidal volume images of the breast.

20. The system of claim 16, wherein using electronic beam steering further comprises acquiring image data of spatially compounded trapezoidal volume images of the breast.

21. The system of claim 16, wherein the ultrasound transducer array is acoustically coupled to the second plate.

22. The system of claim 16, wherein the means for moving the ultrasound transducer array along the path further includes moving the transducer by articulation of the transducer in two dimensions.

23. The system of claim 16, wherein the path includes two passes along an overlapping center portion of the breast.

24. The system of claim 16, wherein the first compression plate and the second compression plates are substantially parallel during compression of the breast.

25. The system of claim 16, wherein the ultrasound transducer array comprises a two-dimensional matrix transducer array.

26. The system of claim 16, wherein the ultrasound transducer array comprises a linear transducer array.

27. The system of claim 16, further comprising:
a processor for processing the image data to form a three dimensional representation of the breast.

28. The system of claim 16, further comprising:
means for transmitting the image data to a location remote from the acquisition location, and
a processor for processing of the transmitted image data to form a three dimensional representation of the breast.

29. The system of claim 16, wherein the moving means moves the ultrasound transducer array by automatically translating the ultrasound transducer array parallel to the second plate in a direction perpendicular to an image plane of the acquired image data.

30. A diagnostic ultrasound imaging system for performing enhanced diagnostic breast imaging, comprising:
a first compression plate;
a second compression plate, the first and second plates being configured for receiving a breast and adapted for compressing the breast between the first and second plates, the breast extending from a chest wall of a patient at a proximate end to a nipple at a distal end, further wherein portions of the breast proximate the nipple and proximate lateral edges of the breast are in non-contact with the second compression plate during breast compression;
an ultrasound transducer array disposed adjacent a side of the second plate opposite the breast, the ultrasound transducer array further being acoustically coupled to the second plate;
translation stage for moving the ultrasound transducer array along a path to scan the breast; and
a controller for acquiring image data representative of the breast as the ultrasound transducer array traverses the path, wherein the controller using electronic beam steering with the ultrasound transducer array to acquire image data in either or both (i) a portion of the breast proximate the chest wall and (ii) a portion of the breast in non-contact with the second plate, and wherein acquiring image data further includes electronic beam steering configured for a one or more pass, three dimensional scan that acquires at least one selected from the group consisting of (a) spatially steered trapezoidal linear image data and (b) spatially steered and compounded trapezoidal linear image data.
